(19) United States
(12) Patent Application Publication
(10) Pub. No.: US 2004/0037900 A1
(43) Pub. Date: Feb. 26, 2004

(54) Shaving gel formulation and methods of use

(76) Inventor: Mack E. Smith, Houston, TX (US)

Correspondence Address: MARTIN L. MCGREGOR, MCGREGOR & ASSOCIATES, 26415 OAK RIDGE DRIVE, SPRING, TX, 77380, US

(21) Appl. No.: 10/224,738

(22) Filed: Aug. 21, 2002

(51) Int. Cl. A61K 35/78; A61K 31/728

(52) U.S. Cl. 424/744; 424/725; 514/54

(57) ABSTRACT

Shaving compositions comprising 25 to 35% aloe vera gel, 4 to 6% propylene glycol, 4 to 6% glycerin, 2 to 4% sodium PCA, 1 to 5% panthenol, 1 to 3% carbomer, 1 to 3% comfrey extract, 1 to 3% of sodium hyaluronate, 1 to 3% of a polyquarternium-10, 0.1 to 0.3% of diazlidinyl urea, 0.4 to 0.6% allantoin and 0.4 to 0.6% triethanolamine, and the balance consisting essentially of de-ionized water and a method of use are disclosed.

### TECHNICAL FIELD

The present invention is related to new gel compositions that have improved lubricating, irritation reduction and conditioning characteristics for shaving and personal care uses.

### BACKGROUND OF THE INVENTION

Personal care products using individual ingredients of the present formulations are known in the personal products area and the individual ingredients have been used in mixtures as shaving cremes as well as other cleansing products. One U.S. Pat. No. 5,653,970 to Vermeer, was located that lists each of the ingredients in various lists of ingredients suitable for use in personal care products that also include heteroatom containing alkyl aldonamide compounds. Numerous references showing individual components in various shaving products are known, however no reference was found that discloses the present relative proportions of the claimed composition as taught herein, nor any reference that suggests the unexpectedly superior properties of the present invention over alternative formulations containing similar ingredients.

### SUMMARY OF THE INVENTION

The present invention provides a composition comprising 25 to 35% aloe vera gel, 4 to 6% propylene glycol, 4 to 6% glycerin, 2 to 4% sodium PCA, 1 to 5% panthenol, 1 to 3% carbomer 1 to 3% comfrey extract, 1 to 3% of sodium hyaluronate, 1 to 3% of polyquarternium-10, 0.1 to 0.3% of diazlidinyl urea, 0.4 to 0.6% allantoin and 0.4 to 0.6% triethanolamine and the balance de-ionized water and optional additional ingredients. A preferred composition further comprises 0.1 to 0.3% a preservative, especially preferred compositions further comprises 0.1 to 0.3% methyl paraben. An alternate preferred composition further comprises 0.05 to 0.15% propylparaben. Another preferred composition further comprises an effective amount of a cosmetically acceptable colorant, an effective amount of a cosmetically acceptable fragrance or both. Preferred colors are FD&C yellow #5 and green in the range of 0.02 to 0.10%. Peach is a preferred fragrance at 0.2 to 0.7%. An especially preferred composition consists essentially of 25 to 35% aloe vera gel, 4 to 6% propylene glycol, 4 to 6% glycerin, 2 to 4% sodium PCA, 1 to 5% panthenol, 1 to 3% carbomer 1 to 3% comfrey extract, 1 to 3% of sodium hyaluronate salt, 1 to 3% of polyquarternium-10, 0.1 to 0.3% of diazlidinyl urea, 0.4 to 0.6% allantoin and 0.4. To 0.6% triethanolamine, 0.1 to 0.3% methyl paraben, 0.05 to 0.15% propylparaben and the balance consisting essentially of de-ionized water Preferably this composition further consists of an effective amount of a cosmetically acceptable colorant, an effective amount of cosmetically acceptable fragrance or both. The most preferred composition consists of 30% aloe vera gel, 5% propylene glycol, 5% glycerin, 3% sodium PCA, 2.5% panthenol, 2% carbomer, 2% comfrey extract, 2% of sodium hyaluronate salt, 2% of polyquarternium-10, 0.2% of diazlidinyl urea, 0.5% allantoin and 0.5% triethanolamine and the balance de-ionized water A preferred composition further consisting of 0.1 to 0.3% a preservative , especially preferred compositions further consisting of 0.2% methyl paraben. An alternate preferred composition further consisting of 0.1% propylparaben. Another preferred composition further consisting of an effective amount of a cosmetically acceptable colorant, an effective amount of a cosmetically acceptable fragrance or both. The current best mode composition consists of the combination of all the foregoing. The preferred colorants are FDA approved.

### DETAILED DESCRIPTION OF THE INVENTION

The inventor has sensitive skin and shaving was for many years a dreaded morning task. Experiments were begun to formulate a superior shaving product that reduced the skin irritation that he usually experienced in shaving. Many formulations were tested by the inventor comprising the preferred ingredients and alternative ingredients. The initial composition tested by the inventor was composed of Water, Aloe Vera, propylene glycol, polysorbate-20, glycerin, triethanolamine, carbomer, diazolidnyl urea, octyl methoxcinnamate, zinc chelazone, methylparaben, allantoin, panthenol, comprey extract, hyaluronic acid, sodium PCA, fragrance, propylparaben, tocopheryl acetate, retinyl palmitate, and FDC colors which were combined in various proportion and tested by the inventor by shaving himself with the materials. The objective was to produce a formulation that improved over commercially available formulations in lubrication and relieving itch and razor burn while at the same time conditioning and smoothing the skin. While the preceding combination of ingredients was in some respects superior to some commercial formulations, it did not in all respects accomplish the desired degree of lubrication, irritation reduction and conditioning.

A later formulation composed of aloe vera, propylene glycol, polysorbate-20, glycerin, triethanolamine, zinc, allantoin, comfrey extract, hyaluronic acid, sodium PCA, pantenol, tocopheryl acetate (Vitamin E), retinyl palmatate (an A vitamin), 2% polyquaternium-10, and preservatives methyl/propylparabens was then tested. While this formulation was useable, it also did not give the desired degree of lubrication. The formulation was altered to the present ingredient ranges after testing various mixtures of the above listed ingredients, over a period of two to three years, the composition was advanced to the currently preferred combination. The currently preferred formulation consisting of 30% aloe vera gel, 5% propylene glycol, 5% glycerin, 3% sodium PCA, 2.5% panthenol, 2% carbomer 2% comfrey extract, 2% of sodium hyaluronate, 2% of polyquarternium-10, 0.2% of diazlidinyl urea, 0.5% allantoin and 0.5% triethanolamine and the balance consisting essentially of de-ionized water. This composition was tested by the inventor and his business associates and found to provide superior qualities over the previous composition. In the inventor's experience, the preferred formulation was found to be superior to currently commercially available shaving gels in protection against razor burn and skin irritation. When the proportions of various ingredients are tested compositions having ingredients within the following ranges provide gels having the desired qualities: 25 to 35% aloe vera gel, 4 to 6% propylene glycol, 4 to 6% glycerin, 2 to 4% sodium PCA, 1 to 5% panthenol, 1 to 3% carbomer 1 to 3% comfrey extract, 1 to 3% of sodium hyaluronate, 1 to 3% of polyquarternium-10, 0.1 to 0.3% of diazlidinyl urea, 0.4 to 0.6% allantoin and 0.4. to 0.6% triethanolamine, 0.1 to 0.3% methyl paraben, 0.05 to 0.15% propylparaben, and the balance consisting essentially of deionized water and optional ingredients. The formulations of the invention may be used with any cosmetically acceptable colors and fragrances. The invention may be varied as disclosed in the summary of the invention above. These experimental examples are given to illustrate the inventive composition claimed herein and are not intended to limit the scope to the invention as set out in the claims below.

We claim:

1. A composition comprising 25 to 35% aloe vera gel, 4 to 6% propylene glycol, 4 to 6% glycerin, 2 to 4% sodium PCA, 1 to 5% panthenol, 1 to 3% carbomer, 1 to 3% comfrey extract, 1 to 3% of sodium hyaluronate, 1 to 3% of polyquarternium-10, 0.1 to 0.3% of diazlidinyl urea, 0.4 to 0.6% allantoin and 0.4. to 0.6% triethanolamine and the balance of de-ionized water.

2. A composition according to claim 1 further comprising 0.1 to 0.3% of a preservative.

3. A composition according to claim 1 further comprising 0.1 to 0.3% methyl paraben.

4. A composition according to claim 1 further comprising 0.05 to 0.15% propylparaben.

5. A composition according to claim 1 further comprising an effective amount of cosmetically acceptable colorant, an effective amount of a cosmetically acceptable fragrance or both.

6. A composition of matter consisting essentially of 25 to 35% aloe vera gel, 4 to 6% propylene glycol, 4 to 6% glycerin, 2 to 4% sodium PCA, 1 to 5% panthenol, 1 to 3% carbomer 1 to 3% comfrey extract, 1 to 3% of sodium hyaluronate, 1 to 3% of polyquarternium-10, 0.1 to 0.3% of diazlidinyl urea, 0.4 to 0.6% allantoin and 0.4 to 0.6% triethanolamine, 0.1 to 0.3% methyl paraben, 0.05 to 0.15% propylparaben and the balance consisting essentially of de-ionized water.

7. A composition according to claim 6 further comprising an effective amount of cosmetically acceptable colorant, an effective amount of a cosmetically acceptable fragrance or both

8. A composition according to claim 6 consisting of 25 to 35% aloe vera gel, 4 to 6% propylene glycol, 4 to 6% glycerin, 2 to 4% sodium PCA, 1 to 5% panthenol, 1 to 3% carbomer, 1 to 3% comfrey extract, 1 to 3% of sodium hyaluronate, 1 to 3% of polyquarternium-10, 0.1 to 0.3% of diazlidinyl urea, 0.4 to 0.6% allantoin and 0.4 to 0.6% triethanolamine, 0.1 to 0.3% methyl paraben, 0.05 to 0.15% propylparaben an effective amount of cosmetically acceptable colorant, an effective amount of a cosmetically acceptable fragrance and the balance consisting essentially of de-ionized water.

9. A composition consisting essentially of 30% aloe vera gel, 5% propylene glycol, 5% glycerin, 3% sodium PCA, 2.5% panthenol, 2% carbomer, 2% comfrey extract, 2% of sodium hyaluronate, 2% of polyquarternium-10, 0.2% of diazlidinyl urea, 0.5% allantoin and 0.5% triethanolamine, 0.2% preservative and the balance consisting essentially of de-ionized water.

10. A composition according to claim 9 further consisting of 0.2% methyl paraben.

11. A composition according to claim 9 further consisting of 0.1% propylparaben.

12. A composition according to claim 9 further consisting of an effective amount of cosmetically acceptable colorant, an effective amount of a cosmetically acceptable fragrance or both

13. A composition according to claim 9 consisting of 30% aloe vera gel, 5% propylene glycol, 5% glycerin, 3% sodium PCA, 2.5% panthenol, 2% carbomer 2% comfrey extract, 2% of sodium hyaluronate, 2% of polyquarternium-10, 0.2% of diazlidinyl urea, 0.5% allantoin and 0.5% triethanolamine, 0.2% methyl paraben, 0.1% propylparaben, an effective amount of cosmetically acceptable colorant, an effective amount of a cosmetically acceptable fragrance and the balance de-ionized water.

14. A composition according to claim 5 wherein the fragrance is peach.

15. A composition according to claim 7 wherein the fragrance is peach.

16. A composition according to claim 9 wherein the fragrance is peach.

17. A composition according to claim 12 wherein the fragrance is peach.

18. A composition according to claim 13 wherein the fragrance is peach.

19. A method for removing hair from the body that comprises applying the composition of claim 1 to the area from which hair is to be removed and then severing the hair with a blade.
